(19) United States
(12) Patent Application Publication
(10) Pub. No.: US 2016/0349239 A1
(43) Pub. Date: Dec. 1, 2016

(54) ELECTRONIC DEVICE AND METHOD FOR DETECTING AND CONTROLLING DRIVING UNDER THE INFLUENCE

(71) Applicant: HON HAI PRECISION INDUSTRY CO., LTD., New Taipei (TW)

(72) Inventor: CHENG-CHING CHIEN, New Taipei (TW)

(21) Appl. No.: 14/794,429

(22) Filed: Jul. 8, 2015

(30) Foreign Application Priority Data
May 29, 2015 (TW) 104117341

(51) Int. Cl. G01N 33/497; G01P 3/00

(52) U.S. Cl. CPC G01N 33/4972; G01P 3/00

(57) ABSTRACT

In a method for detecting and controlling of driving whilst under the influence of alcohol, breath alcohol concentrations of a user are received from an alcohol sensor at predetermined time periods. The breath alcohol concentrations are analyzed to construct a regression curve, and a target time when the user will have become sober can be predicted according to the regression curve. When a dangerous work is done before the target time, an audible warning can be outputted through an output device.

### CROSS-REFERENCE TO RELATED APPLICATIONS

This application claims priority to Taiwanese Patent Application No. 104117341 filed on May 29, 2015, the contents of which are incorporated by reference herein.

### FIELD

The subject matter herein generally relates to driving safety. More particularly, the present disclosure relates to an electronic device and a method for detecting and controlling driving under the influence.

### BACKGROUND

Driving under the influence (DUI), also known as driving while intoxicated (DWI), drunk driving, or impaired driving, among others, is the crime of driving a motor vehicle while impaired by alcohol, to a level that renders a user incapable of operating a motor vehicle safely. DUI increases the risk of accidents.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a block diagram of one embodiment of an electronic device.

FIG. 2 is a block diagram of one embodiment of function modules of a DUI detecting and controlling system.

FIG. 3 is a flowchart of one embodiment of a method for detecting and controlling DUI.

FIG. 4 is an example of a regression curve constructed according to breath alcohol concentrations.

### DETAILED DESCRIPTION

It will be appreciated that for simplicity and clarity of illustration, where appropriate, reference numerals have been repeated among the different figures to indicate corresponding or analogous elements. In addition, numerous specific details are given in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout this disclosure will now be presented.

The word “module,” as used hereinafter, refers to logic embodied in hardware or firmware, or to a collection of software instructions, written in a programming language, such as, for example, Java, C, or assembly. One or more software instructions in the modules may be embedded in firmware. It will be appreciated that modules may comprise connected logic units, such as gates and flip-flops, and may comprise programmable units, such as programmable gate arrays or processors. The modules described herein may be implemented as either software and/or hardware modules and may be stored in any type of non-transitory computer-readable storage medium or other computer storage device. The term “comprising,” when utilized, means “including, but not necessarily limited to”; it specifically indicates open-ended inclusion or membership in the so-described combination, group, series and the like.

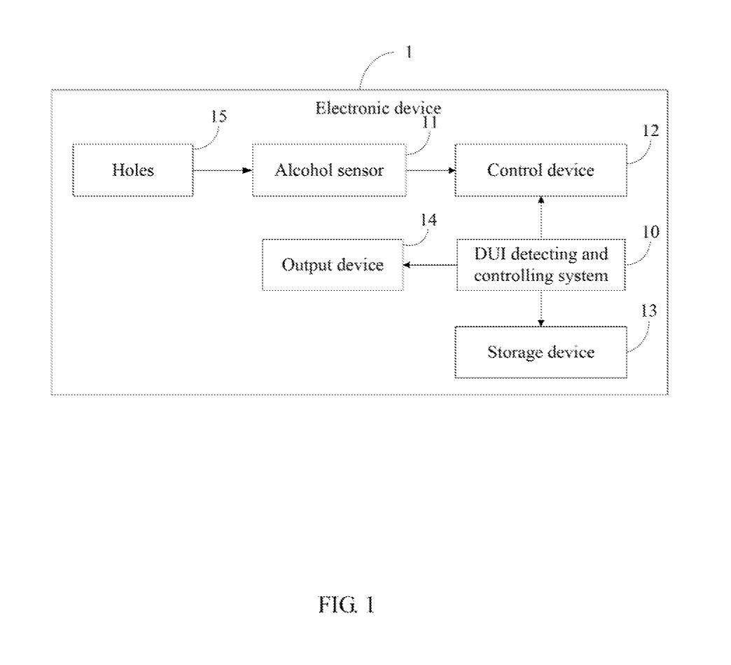

FIG. 1 is a block diagram of one embodiment of an electronic device. The electronic device 1 may be, but is not limited to, a smart phone, a wearable device, a vehicle device, or any other suitable electronic device. The electronic device 1 includes a DUI detecting and controlling system 10 which is designed to prevent someone driving whilst under the influence, or significantly under the influence, of alcohol. In addition, the electronic device 1 further includes, but is not limited to, an alcohol sensor 11, a control device 12, a storage device 13, and an output device 14. FIG. 1 illustrates only one example of the electronic device 1, other examples can include more or fewer components than illustrated, or have a different configuration of the various components in other embodiments.

The DUI detecting and controlling system 10 includes instructions that, when executed by the control device 12, enable the detection of alcohol concentration in the breath of a user, predict a target time when the user can be sober, and output a warning when a dangerous work is done, such as driving occurs, before the time of the user becoming sober. The instructions of the DUI detecting and controlling system 10 can be stored in the storage device 13.

The alcohol sensor 11 can be a gas sensor which detects the presence of gases, such as breath alcohol concentration. In the present application, the alcohol sensor 11 can be a FIGARO sensor.

The control device 12 is electronic circuitry that carries out the instructions of the DUI detecting and controlling system 10, by performing basic arithmetic, logic, and the control and input/output (I/O) operations specified by the instructions. The control device 12 can be central processing unit (CPU), a microprocessor, or other data processor chip.

The storage device 13 can include various types of non-transitory computer-readable storage mediums. For example, the storage device 13 can be an internal storage system, such as a flash memory, a random access memory (RAM) for temporary storage of information, and/or a read-only memory (ROM) for permanent storage of information. The storage device 13 can also be an external storage system, such as a hard disk, a storage card, or a data storage medium.

The output device 14 is any piece of computer hardware equipment used to communicate the results of processing carried out by the DUI detecting and controlling system 10 in human-readable form. In the present embodiment, the output device 14 can be display devices, light emitting diodes (LEDs), loudspeakers, buzzers, and so on.

When the alcohol sensor 11 is located inside of the electronic device 1, the electronic device 1 further include holes 15 which are located above the alcohol sensor 11, enabling the alcohol sensor 11 to fully gather the breath of a user, such as a driver.

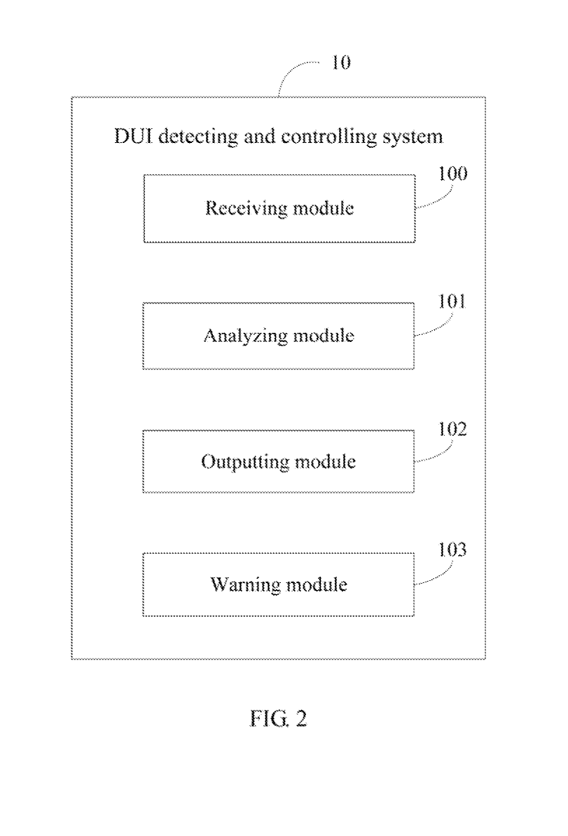

FIG. 2 is a block diagram of one embodiment of function modules of the DUI detecting and controlling system 10. In one embodiment, the function modules of the DUI detecting and controlling system 10 can include a receiving module 100, an analyzing module 101, an output module 102, and a warning module 103.

The receiving module 100 can receive breath alcohol concentrations of a user, analyzed by the alcohol sensor 11, at predetermined time periods. In one embodiment, a user can exhale towards the alcohol sensor 11 at predetermined time periods, such as 10 minutes. The alcohol sensor 11 detects and analyzes alcohol concentrations in the breath, and transmits the alcohol concentrations to the receiving module 100. In other embodiment, the alcohol sensor 11 can actively detects alcohol concentrations in the interior air in the vehicle, for example, at predetermined time periods to obtain the breath alcohol concentrations of a user, and then transmit the alcohol concentrations to the receiving module 100.

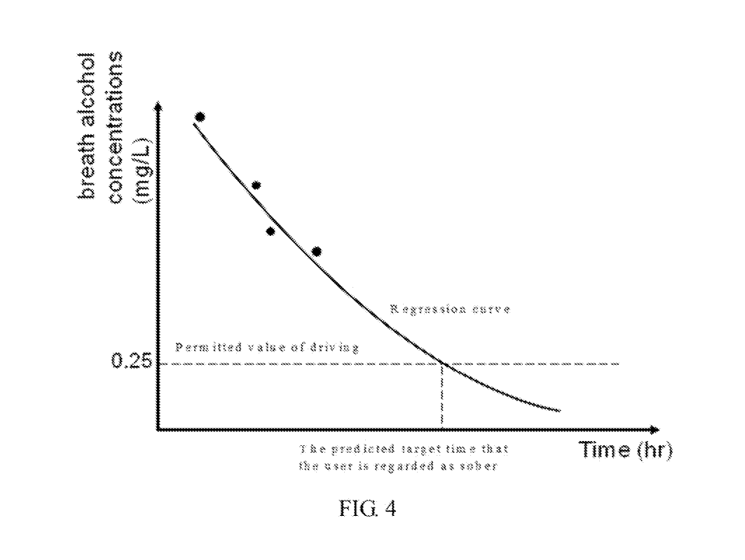

The analyzing module 101 can establish a regression curve according to the breath alcohol concentrations. According to experiments, the concentration of alcohol in the breath of a user can decrease by 0.01-0.05 mg/L per hour according to the constitution, height, weight, and metabolism of the user. Thus, the analyzing module 101 can establish a regression curve according to the breath alcohol concentrations obtained at different points in time, as shown in FIG. 4.

The analyzing module 101 can further predict a target time when the user can be legally sober, according to the regression curve. Referring to FIG. 4, when the breath alcohol concentrations of a user has decreased so as to be equal to or less than a permitted alcohol concentration value for driving, or for doing something else, it is considered that the user has become sober. The permitted alcohol concentration value may be 0.25 for example.

The output module 102 can output the target time through the output device 14, and the warning module 103 can output a warning when a dangerous work is done prior to the target time the user has become sober, through the output device 14. In one embodiment, the output device 14 can include a display device and a loudspeaker. The display device can output the target time, and the loudspeaker can be used to output the warning.

In one embodiment, the electronic device 1 can further include a speed sensor, which can be used to sense movements of the user. When the speed senor senses that a movement speed of the user is greater than a predetermined threshold before the target time, it is determined that the user is doing a dangerous work and the warning module 103 can output a warning through the output device 14.

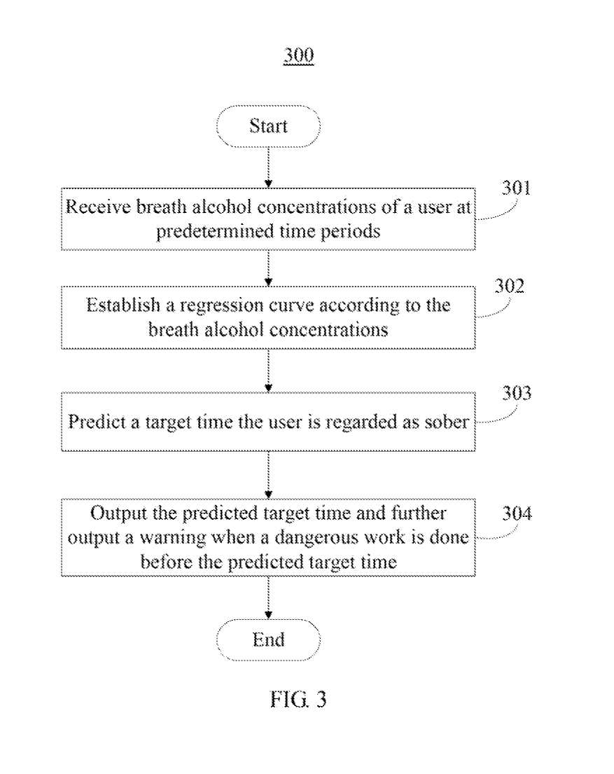

FIG. 3 is a flowchart of one embodiment of a method for detecting and controlling DUI.

Referring to FIG. 3, a flowchart is presented in accordance with an example embodiment. The example method 300 is provided by way of example, as there are a variety of ways to carry out the method. The method 300 described below can be carried out using the configurations illustrated in FIGS. 1 and 2, for example, and various elements of these figures are referenced in explaining example method 300. Each block shown in FIG. 3 represents one or more processes, methods, or subroutines carried out in the exemplary method 300. Furthermore, the illustrated order of blocks is by example only and the order of the blocks can change. Additional blocks may be added or fewer blocks may be utilized, without departing from this disclosure. The exemplary method 300 can begin at block 301.

At block 301, a receiving module receives breath alcohol concentrations of a user from an alcohol sensor at predetermined time periods. In one embodiment, the user can exhale towards the alcohol sensor at predetermined time periods, such as 10 minutes. The alcohol sensor detects and analyzes alcohol concentrations in the breath, and transmits the alcohol concentrations to the receiving module. In other embodiment, the alcohol sensor can actively detects alcohol concentrations in the interior air of a vehicle, for example, at predetermined time periods, to obtain the breath alcohol concentrations of a user, and then transmit the alcohol concentrations to the receiving module 100.

At block 302, an analyzing module establishes a regression curve according to the breath alcohol concentrations. According to experiments, breath alcohol concentrations of a user can decrease by 0.01-0.05 mg/L per hour according to the constitution, height, weight, and metabolism of a user. Thus, the analyzing module can establish a regression curve according to the breath alcohol concentrations obtained at different points in time, as shown in FIG. 4.

At block 303, the analyzing module predicts a target time when the user can be regarded as sober according to the regression curve. Referring to FIG. 4, when the breath alcohol concentrations of the user has decreased to an amount which is equal to, or less than, a permitted value for driving, or for doing something else, it is considered that the user has become sober.

At block 304, an output module outputs the target time through an output device, and a warning module outputs a warning when a dangerous work is done, such as driving occurs, before the target time when the user has become sober, through the output device. In one embodiment, the output device can include a display device and a loudspeaker. The display device can be used to output the target time, and the loudspeaker can be used to output the warning.

In one embodiment, a speed sensor can be used to sense movements of the user. When the speed senor senses that a movement speed of the user is greater than a predetermined threshold, before the target time when the user has become sober, it is determined that the user is doing a dangerous work. In this circumstance, the warning module can output a warning through the output device 14.

The embodiments shown and described above are only examples. Many details are often found in the art. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. A method for detecting and controlling driving under the influence, the method executable by at least one processor of an electronic device and comprising:
receiving breath alcohol concentrations of a user via an alcohol sensor at predetermined time periods;
establishing a regression curve according to the breath alcohol concentrations;
predicting a target time that the user is regarded as sober according to the regression curve; and
outputting a warning via an output device when driving occurs prior to the target time.

2. The method according to claim 1, further comprising:
outputting the target time through the output device.

3. The method according to claim 1, wherein the target time is a time that the breath alcohol concentrations of the user has decreased to an amount which is equal to or less than a permitted value.

4. The method according to claim 1, further comprising:
sensing a movement speed of the user before the target time; and
outputting a warning when the movement speed is greater than a predetermined threshold.

5. An electronic device, comprising:
an output device;
at least one processor; and
a storage storing one or more programs which, when executed by the at least one processor, causes the at least one processor to:
receive breath alcohol concentrations of a user via an alcohol sensor at predetermined time periods;
establish a regression curve according to the breath alcohol concentrations;
predict a target time that the user is regarded as sober according to the regression curve; and
output a warning via an output device when driving occurs prior to the target time.

6. The electronic device according to claim 5, wherein the at least one processor further:
outputs the target time through the output device.

7. The electronic device according to claim 5, wherein the target time is a time that the breath alcohol concentrations of the user has decreased to an amount which is equal to or less than a permitted value.

8. The electronic device according to claim 7, wherein the electronic device further comprises a speed sensor which senses movements of the user; and when sensing that a movement speed of the user is greater than a predetermined threshold before the target time, it is determined that the user is doing a dangerous work.

9. The electronic device according to claim 5, wherein the alcohol sensor is located inside of the electronic device.

10. The electronic device according to claim 9, wherein the electronic device further comprises holes which are located above the alcohol sensor.

11. A non-transitory storage medium having stored thereon instructions that, when executed by at least one processor of an electronic device, causes the at least one processor to perform a method for detecting and controlling of driving under the influence, the method comprising:
receiving breath alcohol concentrations of a user via an alcohol sensor at predetermined time periods;
establishing a regression curve according to the breath alcohol concentrations;
predicting a target time that the user is regarded as sober according to the regression curve; and
outputting a warning via an output device when driving occurs prior to the target time.

12. The non-transitory storage medium according to claim 11, wherein the method further comprises:
outputting the target time through the output device.

13. The non-transitory storage medium according to claim 11, wherein the target time is a time that the breath alcohol concentrations of the user has decreased to an amount which is equal to or less than a permitted value.

14. The non-transitory storage medium according to claim 11, wherein the method further comprises:
sensing a movement speed of the user before the target time; and
outputting a warning when the movement speed is greater than a predetermined threshold.
